(19) United States
(12) Patent Application Publication
(10) Pub. No.: US 2020/0224167 A1
(43) Pub. Date: Jul. 16, 2020

(54) Direct effect of extracted BMP on Mesenchymal Stem Cell Differentiation for use in a Culture Medium, Diagnostic Assay, and Adjunct or Stand-Alone Pharmaceutical Treatment

(71) Applicant: Zycal Bioceuticals, Inc., Toms River, NJ (US)

(72) Inventor: Dan Tripodi, Hazelton, PA (US)

(73) Assignee: ZyCal Bioceuticals, Inc., Toms River, NJ (US)

(21) Appl. No.: 16/708,169

(22) Filed: Dec. 9, 2019

Related U.S. Application Data
(60) Provisional application No. 62/778,546, filed on Dec. 12, 2018.

(51) Int. Cl. C12N 5/077; C12N 5/00

(52) U.S. Cl. CPC C12N 5/0654; C12N 5/0018; C12N 2501/155; C12N 2501/148; C12N 2533/54; C12N 2501/115; C12N 2501/165; C12N 2501/105; C12N 2506/1346; C12N 2501/15

(57) ABSTRACT

The present invention comprises a culture medium composition for the proliferation and differentiation of mesenchymal stem cells into osteoblasts, the composition comprising a bone morphogenic protein (BMP) complex with TGF-f3, bFGF, VEGF, and IGF, bound to collagen type 1 dissolved into a mesenchymal stem cell culture medium. Methods of use of the composition and/or the BMP complex comprise: performing a diagnostic assay detecting a level of bone morphogenetic proteins in a patient serum level; determining if the BMP level is below a threshold level; and when the BMP level is below the threshold level, administering a composition comprising a BMP complex, with or without a pharmaceutical, such as a drug for treating and/or preventing osteoporosis. The present invention comprises performing an in vivo lavage and bone assay; and administering the BMP complex composition to a patient in need thereof.

### CROSS-REFERENCE TO RELATED APPLICATIONS

The Applicant claims the benefit of the filing date of U.S. Application No. 62/778,546 which was filed on Dec. 12, 2018.

### FIELD OF USE

This invention relates generally to the field of tissue engineering; and in particular to a novel culture medium to proliferate and differentiate in vitro mesenchymal stem cells into tissue, such as into osteoblasts for bone tissue formation.

### COPYRIGHT NOTICE

A portion of the disclosure of this provisional patent application document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### Trademarks Disclaimer

The product names used in this document are for identification purposes only. All trademarks and registered trademarks are the property of their respective owners.

### BACKGROUND

Mesenchymal stem cells (MSC) can differentiate into adipocytes, osteoblasts, myocytes, and chondrocytes in vivo and in vitro; and into hepatocytes, neurons and pancreatic islet cells in vitro when specific culture conditions and stimuli are applied (1)(2). The directed differentiation of MSC in vitro requires using appropriate differentiation media and culture conditions, which depend on the type of final cells/tissue desired. Stem cell differentiation is also an unpredictable, difficult laboratory process in which minor modifications in conditions can determine the success or failure of the experiments.

Cyplexinol® is a natural bone morphogenic protein (BMP) complex from the present inventor, which consists of a collagen fragment matrix with the BMPs and endogenous growth factors bound within and to the matrix. Upon oral administration of the BMP-complex, the BMP moieties bind to receptors within the GI lumen where they quickly confer both osteoinductive and anti-inflammatory activity. It is the osteogenic properties of the BMPs which differentiate the MSCs into osteoblasts to promote the growth of de novo bone tissue, providing the critical surface for minerals to bind to healthy bones (3).

Bone from MSCs

Derivatives of bone have long been used in the surgical repair of bone defects and bone trauma in mammals. For example, U.S. Pat. No. 9,173,905 B1 and U.S. Pat. No. 9,011,930 B2, by the inventor of the present application, disclose an orally administered pill or tablet comprising demineralized bone (DMB) that is taken as a nutritional supplement to improve bone and/or cartilage health in a mammal. Both patents are incorporated herein by reference in their entirety.

Subsequent approaches have used bone repair matrices containing bioactive proteins which when implanted into a bone defect provided not only a scaffolding for invasive bone ingrowth, but active induction of bone cell replication and differentiation. These materials are generally referred to as osteoinductive.

In general, osteoinductive compositions comprise a matrix which provides the scaffolding for invasive growth of the bone, anchorage dependent cells, and an osteoinductive protein source. The matrix may be a variety of materials, such as: collagen (Jefferies U.S. Pat. Nos. 4,394,370 and 4,472,840); inorganically based materials, such as a biodegradable porous ceramic (Urist U.S. Pat. No. 4,596,574); or, polylactic acid (Urist U.S. Pat. No. 4,563,489).

### Tissue Engineering

Tissue Engineering is the art of implanting a biocompatible, biodegradable scaffold with seeded stem cells into a patient, wherein the scaffold degrades as the stem cells differentiate into neo-tissue. Unfortunately, bone tissue engineering to date has failed to reliably produce bone matrix that is able to withstand normal mechanical forces applied to the human skeleton (e.g. sheer force, compressive loads, etc.).

Mesenchymal stem cells (MSC) are fibroblastoid multipotent adult stem cells with a high capacity for self-renewal; and they have been isolated from several human tissues, including bone marrow, adipose tissue, umbilical cord matrix, tendon, lung, and the periosteum. Various MSC culture conditions are well known in the art of tissue engineering to induce MSCs perfused with osteogenic culture medium to differentiate in vitro to a specific cell type, e.g. osteoblasts, versus chondrocytes. Not only the culture medium, and the growth factors and other nutrients added to the medium, are crucial in effectively differentiating the MSCs to the desired tissue/cell type. Other culture conditions (e.g. temperature, gases, cell contact surface, cell manipulation, etc.) also play a key role in the success of the protocol.

The standard osteogenic culture medium for proliferating and differentiating mesenchymal stem cells (MSCs) to bone osteoblasts in vitro comprises dexamethasone (Dex), ascorbic acid (Asc) and β-glycerophosphate (β-Gly); however, the effectiveness of these osteogenic factors in vivo (such as in tissue engineering when a scaffold with MSCs are implanted) is less clear; and MSCs pre-treated with dexamethasone is not suitable in vivo for use in treating a patient with an immunological disorder (4).

The osteogenic differentiating of MSCs in vitro is divided into three stages. In stage one (e.g. day 1 to 4), a peak in the number of cells occurs. In stage two, from day 5 to 14, early cell differentiation of MSCs to osteoblasts occurs, which is characterized by transcription and protein expression of alkaline phosphatase (ALP), after which the ALP declines; and collagen type 1 expression. In stage three, from day 14 to 28, the cells express high levels of osteocalcin and osteopontin, followed by calcium and phosphate deposition (4). The success of the differentiation of MSCs to osteoblasts can be studied by screening for the presence of these and other biomarkers.

What is needed within the tissue engineering field is a more robust and predictable mesenchymal stem cell culture medium that reliably produces osteoblasts, osteocytes, bone. The MSCs culture medium should be able to be transplanted in vivo in a mammal (with and without a bio-scaffold-matrix) to produce bone able to handle normal mechanical loads in vivo; and/or to produce bone (or stage 1, 2, or 3 of the differentiation process), which can then be implanted to differentiate into full bone.

Additionally, a MSC culture medium with demineralized bone matrix in needed to study in vitro the differentiation process (e.g. cell signaling, effect of various growth factors on cell differentiation, etc.) in a cell culture dish, flask, or the like (e.g. a bioreactor). This medium can be used, especially in the absence of dexamethasone, to further determine the impact of bone osteoblasts and osteocytes, and the endogenous growth factors they produce, have on the differentiation process (such as in vivo).

### SUMMARY OF THE INVENTION

In an embodiment, the present invention is directed to two different compositions (non-differentiation versus osteogenic) useful as in vitro culture mediums to proliferate and differentiate, in a culture dish or the like, mesenchymal stem cells (MSCs) into bone matrix (i.e. tissue).

In an embodiment, the mesenchymal stem cell differentiation to osteoblasts than to osteocytes (e.g. bone) occurs more quickly and/or produces more dense bone matrix with the Cyplexinol® powder (i.e. as disclosed in U.S. Pat. No. 9,173,905 B1 and U.S. Pat. No. 9,011,930 B2 by the inventor of the present application) added to a cell differentiation culture medium, than without it added, such as for used in tissue engineering in vitro experiments.

In an embodiment, the present invention comprises a composition comprising the culture mediums disclosed herein, and a method of making the culture mediums, by adding demineralized bone matrix and/or bone morphogenic protein (BMP) COMPLEX (e.g.

Cyplexinol®) to a culture medium known in the art for use with MSCs differentiating to bone, such as to Lonza™ Mesenchymal stem cell (MSC) Culture Medium (e.g. Lonza Non-differentiating culture medium; or Lonza Osteogenic Differentiating medium).

In an embodiment, the culture medium to which the demineralized bone matrix and/or BMP COMPLEX) (e.g. Cyplexinol®) is added does not comprise dexamethasone (e.g. Lonza Non-differentiating culture medium). This composition produces more dense bone, and more quickly, than the second osteogenic composition or a control lacking Cyplexinol.

In another embodiment, the culture medium to which the Cyplexinol is added is Lonza Osteogenic Differentiating medium, which comprises: dexamethasone, L-glutamine, ascorbate, penicillin/streptomycin, MCGS, and beta-glycerophosphate.

In one embodiment, the composition further comprises a biocompatible lattice or scaffold comprising one composition of the present invention.

In another embodiment, the present invention comprises a method of expanding a population of mesenchymal stem cells in vitro.

In another embodiment, the present invention comprises a method for differentiating mesenchymal stem cells into bone cells/tissue, which comprises culturing the mesenchymal stem cells in a culture medium containing a Cyplexinol® BMP complex comprising bovine BMP's, active growth factors TGF-β, bFGF, VEGF, and IGF, bound to collagen.

In another embodiment, the present invention comprises various methods of use of the culture mediums disclosed herein to study in vitro MSC's differentiation pathways (e.g. the effect of culture conditions, growth factors added, temperature, etc.; bone versus cartilage differentiation based on culture conditions). Studies can be performed with the compositions used in the culture dishes, or the similar experimental labware (e.g. flasks); and/or studies can be performed in bioreactors with culture medium perfused, with or without a scaffold.

In another embodiment, the present invention comprises a method of treatment for bone repair in a mammal by using a composition comprising the culture medium disclosed herein to proliferate and differentiate MSCs into bone, or bone and cartilage in vitro, which is subsequently implanted in vivo into a mammalian bone defect (e.g. within a long or short bone fracture; a joint-knee, shoulder, ankle, etc.).

In another embodiment, the method of treatment comprises inserting the MSCs into a biocompatible, biodegradable scaffold, perfusing the scaffold with the composition of the present invention (e.g. in vitro via the use of a bioreactor or a laminar flow hood), and implanting the scaffold into the patient's bone defect in vivo. The surgical site may further be perfused with the composition of the present invention.

In another embodiment, the culture medium composition comprising BMP's complex (e.g. Cyplexinol®) can further be used for mammalian in vivo differentiation either in conjunction with transplantation of heterologous or homologous, stem cells, or to augment naturally occurring differentiation with endogenous stem cells, or to act as an adjunct to various pharmaceuticals designed to promote stem cell function.

In another embodiment, the present invention comprises a diagnostic assay for detecting bone morphogenetic proteins (BMP) serum levels, and then administering MSC compositions of the present invention comprising demineralized bone matrix (e.g. Cyplexinol®) when BMP levels fall below a threshold level.

In another embodiment, the present invention comprises a method of treatment using in vivo lavage and bone assay.

In another embodiment, the present invention comprises a pharmaceutical adjunct to existing treatments and/or compositions for bone related disorders, wherein demineralized bone matrix is co-administered or separately administered with other compositions and/or treatments.

Other aspects of the invention will become apparent by consideration of the detailed description.

### BRIEF DESCRIPTION OF THE DRAWINGS

The above and other features, aspects and advantages of various embodiments will become better understood with regard to the following description, appended claims, and accompanying drawings where:

FIG. 1 is a schematic of the setup of the culture dishes for the Protocol Experimentation 1.

FIG. 2 are images of the results Alizarin red staining of four different compositions (replicated) after 14 days of culturing with 6,200 MSCs in each well; wherein the greatest amount of differentiation of MSC's to osteoblasts is shown by the red dots in the box 2's staining the osteoblast's bone mineral, which comprise the 100 mg/ml of Cyplexinol® with Lonza™ Non-Differentiating Culture Medium.

FIG. 3 are images of the results Alizarin red staining of four different compositions (replicated) of FIG. 2 after 21 days of culturing with 6,200 MSCs in each well.

FIG. 4 illustrates the change in ALPL gene expression levels.

FIG. 5 illustrates the change in BGLAP gene expression levels.

FIG. 6 illustrates the change in GLI1 gene expression levels.

FIG. 7 illustrates the change in Sp7 gene expression levels.

### DETAILED DESCRIPTION

### Glossary of Terms

Bone: bone used herein is recovered from any source including animal and human. Such bone includes any bone or portion thereof, including cut pieces of bone, including cortical and/or cancellous bone, for example, recovered from a human or animal. Such bones include for example, the humorous, hemi-pelvi, tibia, fibula, radius, ulna, rib, vertebrae, mandibular, femur, and, ilia, and any cut portion thereof, and also including continuous or discontinuous bone portions. When referred to generally, such bone includes demineralized and not demineralized bone. In a preferred embodiment cancellous or cortical bone material is demineralized. For purposes of the present invention all such forms of bone include one or more therapeutically beneficial substances including, for example, at least one of bone morphogenetic protein and/or transforming growth factor-β.

Bone Morphogenetic Protein (BMP): BMP comprises a family of proteins and has been designated or identified as BMP-1 through BMP-8, inclusive, as disclosed in U.S. Pat. Nos. 4,877,864; 5,013,649; WO 90/11366; and WO 91/18098, as well as BMP-9, BMP-10, BMP-11, BMP-12 and BMP-13. These substances are further described and disclosed hereinbelow. Such proteins can be extracted from demineralized bone matrix (BMP COMPLEX) by methods known in the art and used apart from the demineralized bone. A preferred BMP is BMP-2, the mature protein sequence beginning with the amino acid Gln at nucleotide 1202 and ending with the amino acid Arg at nucleotide 1543, as described in detail in U.S. Pat. No. 5,013,649. Combinations of two or more of such osteogenic proteins are suitable for use in the present invention, as are fragments and heterodimeric forms of such proteins that exhibit osteogenic activity.

Cyplexinol® BMP Complex-Mechanism of Action: Cyplexinol® is a BMP-Complex that is extracted from certified organic, closed-herd, bovine femurs. The hydroxyapatite component of the bone is removed, leaving behind the native, partially hydrolyzed collagenous network of type 1 collagen and associated growth factors, e.g. TGF-β, IGF, bFGF, VEGF, and isoforms of BMPs. The partially hydrolyzed collagen protects the active proteins and ensures their delivery to receptors in the Gastrointestinal mucosa, where they confer their osteoinductive activity upon MSCs. In particular, the tertiary conformation of the proteins and collagen resists enzymatic and hydrolytic degradation. Furthermore, bioavailability studies have demonstrated that the target specificity of BMP can be attributed to the presence of BMP-7 receptors in the stomach and ileum of animals, which transport the active peptide rather than peptides that have been hydrolyzed into amino acids. The presence of additional BMP receptors, in the gastrointestinal system have also been identified through animal models. Accordingly, BMP-2, BMP-4, and BMP-7 are expressed in gastrointestinal tissues, where they have been shown to play a significant role in the regulation of cellular proliferation and differentiation. Studies have also indicated that Smad 4, which forms a heteromeric complex with Smads 1, 5, and 8 after they are phosphorylated by BMP-activated type I receptor, is localized in the gastrointestinal system. Therefore, the BMP signaling pathway facilitates the delivery and absorption of biologically active BMPs. Once the osteoinductive properties of the BMPs within the Cyplexinol® complex have been conveyed through the GI receptors, the osteogenic differentiation of MSCs into osteoblasts occurs, followed by the growth of de novo bone tissue and mineralization. Similarly, the osteoinductive proteins that comprise Cyplexinol® have also been proven to turn MSCs into chondrocytes for new cartilage tissue growth via proteoglycan excretion. However, in addition to facilitating the proper cellular groundwork for tissue regeneration, BMP activation can have a rapid response on pain signaling in the joint due to interleukin regulation/feedback. The proliferation of inflammatory cytokines, such as IL-1 and IL-6, within the joint space can have detrimental effects on joint tissue. IL-1, in particular, activates MMPs and the NFKappa B transcription factor, while elevated IL-6 levels leads to chronic inflammation. Accordingly, Cyplexinol® has been shown to downregulate the activation of pro-inflammatory cytokines such as IL-1 and IL-6. More specifically, this natural ingredient demonstrated the ability to disrupt the IL-1 and IL-6 inflammatory pathways in as little as seven days. This positions Cyplexinol® as a novel ingredient that elicits a rapid anti-inflammatory effect. More importantly, the BMP of complex, Cyplexinol®, disrupts inflammatory pathways that would otherwise augment joint damage and in doing so, promotes joint health (1).

Collagen: the protein substance of the white fibers (collagenous fibers) of skin, tendon, bone, cartilage and all other connective tissue, composed of molecules of tropocollagen, it is converted into gelatin by boiling. The term collagenous pertains to collagen, forming or producing collagen. Collagen is distinguished from bone by those skilled in the art, particularly relating to bone and collagen derived compositions useful for bone repair. For example, U.S. Pat. No. 4,440,750 discloses a two component composition that is used for bone repair or construction, “(p)articulate demineralized bone and reconstituted collagen are the two principal components of the composition.” (col. 1, lns. 63-65).

Demineralized Bone: one or more distinct bone portions which have been demineralized by any method well known to those of ordinary skill in the art. Typically, cortical and cancellous bone are demineralized in hydrochloric acid for a period of time of about 15 minutes to about 8 hours or more at temperatures ranging from less than ambient, e.g., greater than about 0° C. to about 22° C. to temperatures slightly to moderately elevated above ambient, e.g., about 25° C. to about 50° C. Typically, cortical and/or cancellous bone is demineralized to contain less than about 10 wt % residual calcium; preferably about less than about 5 wt % residual calcium; more preferably about 1 wt % to about 3 wt % calcium; even more preferably about 2 wt % residual calcium or less; for example, containing trace amounts to about 2 wt %. Other methods for demineralizing bone are well known in the art to which the present invention pertains, and can be readily selected and employed by one of ordinary skill in the art, without undue experimentation. Further detailed descriptions of suitable methods are set forth below. When bone is suitably demineralized and in particulate form the resulting material can also be referred to as demineralized bone matrix (BMP COMPLEX) or demineralized bone powder. A BMP COMPLEX suitable for use in the present invention comprises substances such as bone morphogenetic protein (BMP) described above, collagen type I and at least one chondroblast or osteoblast stimulating growth factor. It is known that the major collagen of skin, tendon, and bone is the same protein containing two alpha-1 polypeptide chains and one alpha-2 chain. Osteoblast stimulating growth factor is also referred to as insulin-like growth factor I or IGF-I; it is known to induce various cellular activities, including bone growth. A chondroblast is a cell that arises from the mesenchyma and forms cartilage. Osteoblast stimulating growth factor comprises at least one substance selected from the group consisting of transforming growth factors-beta (TGF-β), such as TGF-β1 and TGF-β2, BMP-2 through BMP-13, inclusive, insulin-like growth factor (IGF), including IGF-I and IGF-II, platelet-derived growth factor (PDGF), including PDGF AA, PDGF BB and PDGF AB, and fibroblast growth factors (FGF), particularly basic-FGF or FGF2. The osteoblast stimulating characteristics of a substance can be characterized, for example, by observation of increased proliferation of an osteoblastic cell line in culture, including a cell line selected from the group consisting of MC3T3-E 1, AsOS2, TE85 and MG63. Alternatively, osteoblast stimulation can be measured by an altered expression of osteoblastic markers, e.g., alkaline phosphatase, osteocalcin and osteopontin. Chondroblastic stimulation can be measured by increased rate of proliferation of a cultured chondroblastic cell line, such as in a cell line selected from the group consisting of HTB-94, TMC23, and ATDCS. Alternatively, chondroblast stimulation can be measured by altered expression of a chondroblastic marker such as collagen II, collagen X or hyaluronic acid, in cultured chondrocytic cells.

Mammal: for purposes of the present invention mammal refers to any animal classified as a mammal, including humans, domestic and farm animals, and zoo, sports, or pet animals, such as dogs, horses, cats, cows, etc. Preferably, the mammal is human.

Osseous tissue: also referred to as bone tissue. It is a connective tissue, the matrix of which consists of collagen fibres and ground substance and in which are deposited calcium salts (phosphate, carbonate, and some fluoride) in the form of an apatite mineral. Prior to calcification, osseous tissue is referred to as osteoid tissue. It is uncalcified bone matrix that is produced in the body by osteoblasts. It consists mainly of collagen, but has osteonectin present.

Osteoconductivity (or osteoconductive): the ability of a substance to serve as a scaffold for bone growth. Osteoconductive materials are typically biocompatible matrix materials, for example, hydroxyapitate; collagen; biocompatible matrix materials including for example, polymeric matrix materials, bioglass, bioceramics, resorbable biomaterials, bioabsorbable polymers, a plastic matrix, stainless steel, titanium, and cobalt-chromium-molybdenum alloy matrix; and commercially available, synthetically prepared substances that include hydroxyapitate.

Osteogenic: commonly refers to bone formation by living cells.

Osteoinductivity (or osteoinductive): the ability of a substance to induce osteoblast differentiation for the promotion of bone growth. Osteoinductive substances include but are not limited to, for example, autograft bone; allograft bone; commercially available synthetic grafting compositions; demineralized cortical bone, demineralized cancellous bone and collagen, and mixtures thereof. For suitable use in the present invention, such substances comprise or include one or more growth factors, specifically, osteoinductive growth factors. Such growth factors include for example, bone morphogenetic protein (BMP) and transforming growth factor-beta (TGF-β, see below). Osteoinductive substances can be characterized, for example, by their ability to alter the expression of markers associated with osteoblasts, chondroblasts, osteocytes, or chondrocytes in cultured cells. Such markers include, for example, with regard to osteoblast stimulation, alkaline phosphatase, osteocalcin, osteopontin; and with regard to chondroblast stimulation, collagen II, collagen X, and hyaluronic acid in cultured chondrocytic cells. As described in further detail in the present disclosure, a demineralized bone product useful in the present invention is typically demineralized to the extent that it comprises less than about 6 wt % residual calcium; preferably comprising about 1 wt % to about 3 wt % residual calcium; more preferably comprising about 2 wt % residual calcium or less; for example, comprising trace amounts to about 2 wt %.

TGF-beta: Transforming Growth Factors-0 (TGF-β) refers to multifunctional peptides that control proliferation, differentiation and other functions in many cell types, including bone. It is reportedly a potent stimulator of osteoblastic bone formation. TGF-0 is the prototype of a protein family also known as the TGF-β superfamily. The family includes inhibin A and B, activin-A, B and AB, Mullerian inhibiting substance, bone morphogenetic proteins, decapentaplegic and vegetalising factor-1. The TGF-β superfamily may comprise as many as 100 distinct proteins. TGF-β exists in at least five isoforms known as TGF-β-1 through TGF-β-5 inclusive; TGF-β-1 is the prevalent form. Mature human, porcine, simian, chicken and bovine TGF-0-1 are identical.

### Method of Making Culture Medium

The following is an exemplification of a method of making an MSC culture medium using BMP complex, of which one skilled in the art would now of other MSC culture mediums to use.

Step 1: the method of making the composition for MSC differentiation first requires preparing the Cyplexinol® powder. In an embodiment, the BMP complex comprises endogenous bone morphogenic proteins (BMPs) with growth factor proteins (e.g. Transforming Growth Factor-beta) that survive the demineralization process intact; and are active proteins able to facilitate or improve the differentiation of MSCs into bone, and/or bone and cartilage.

Step 2: comprises providing a stem cell culture medium suitable for differentiating MSCs into bone matrix. In an embodiment, the stem cell culture medium comprises: Lonza™ Non-differentiation Medium; or Lonza™ Osteogenic Medium.

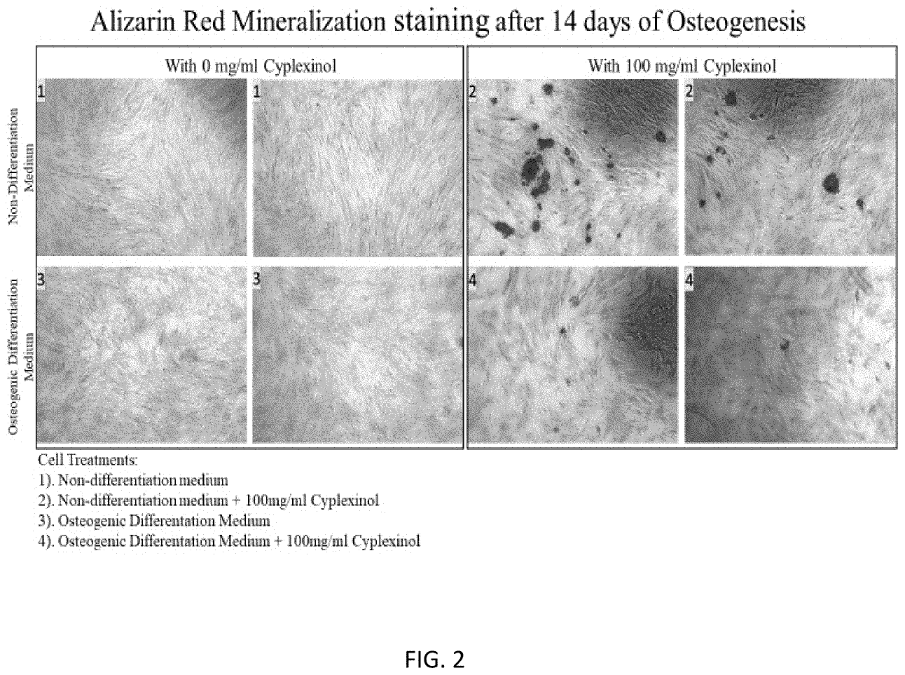

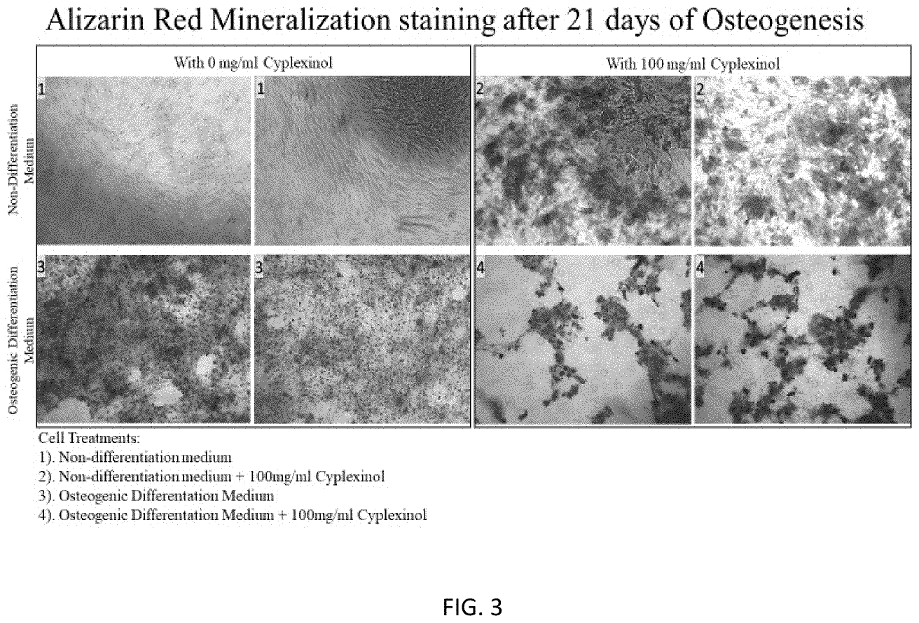

Step 3 in the method of making the composition: comprises dissolving the Cyplexinol® DMB power into the Lonza™ Non-differentiation Medium, or the Lonza™ Osteogenic Medium, in accordance with the protocol of Table 1. The optimal amount of Cyplexinol® powder to add to the Lonza culture medium (as determined by Experiment 1, infra, and the resulting maximum amount of MSCs that differentiated into osteoblasts per FIGS. 2 and 3 images) was determined to be 100 mg/ml of Cyplexinol. This equates to (4 g Cyplexinol/37 ml of Lonza medium) to (1 g Cyplexinol/9.25 ml of Lonza medium).

TABLE 1
Protocol for Dissolving Cyplexinol

| | |
|---|---|
| 1. | 100 mg/ml of Cyplexinol added to Lonza Mesenchymal stem cell |
| | (MSC) medium (Non-differentiating or Osteogenic Differentiating) |
| | Example: |
| | Add weight 4 g of Cyplexinol in a 50 ml centrifuge tube. |
| | Add medium to 40 ml mark, which equals to about 37 ml |
| 2. | Rock the medium and Cyplexinol for 2 hours at 4° C. |
| 3. | Centrifuge the medium at 5,000 × g for 5 minutes at 15′ C. to pellet |
| | the undissolved compound |
| 4. | Collect supernatant in sterile syringe |
| 5. | Repeat centrifuging and collecting supernatant 2-3 more times |
| 6. | Pass the medium supernatant through a 0.2.2pm PES syringe filter |

### Exemplification 1—Determining Cyplexinol with Lonza Culture Medium Concentration

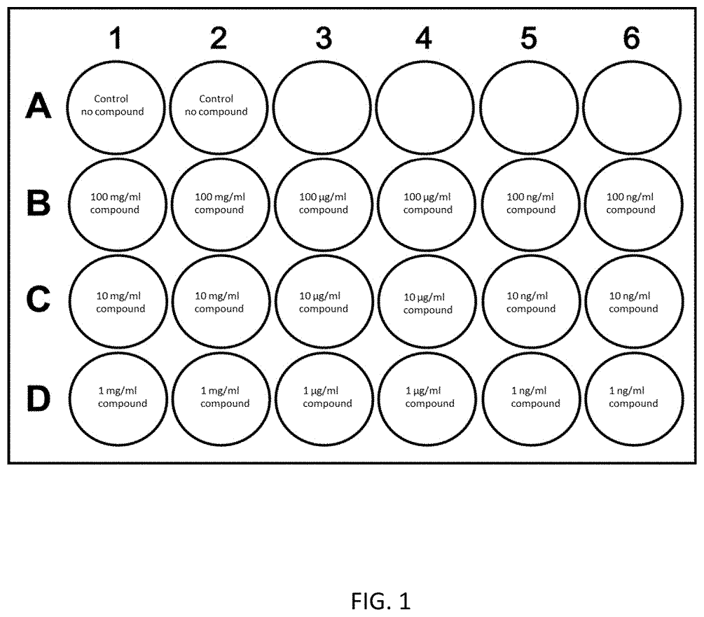

As illustrated in FIGS. 1-7, and Table 1, a pilot experiment was performed to determine the optimal amount of composition of the present invention that differentiates the maximum amount of MSCs to bone matrix. A control or a different composition was added to each well of a 24 well plate, with each well comprising 6,200 MSCs. Duplicates of the following amounts of Cyplexinol were added to the Lonza Non-differentiating medium, and the Osteogenic Medium (not shown in FIG. 1): 100 mg/ml; 10 mg/ml; 1 mg/ml; 100 μg/ml; 10 μg/ml; 1 μg/ml; 100 ng/ml; 10 ng/ml; and 1 ng/ml of Cyplexinol DMB powder. The control wells comprised Lonza Non-differentiating medium with MSCs and without Cyplexinol. The MSCs were cultured for 28 days, and the composition (e.g. the culture medium) was changed every 72 hours.

Alizarin red staining is widely known in the art of tissue engineering to mark the extracellular calcium deposits in mineralized bone matrix within mature osteoblasts. At day 21 (FIG. 2), and at day 28 (FIG. 3) of the culture, Alizarin red staining was performed on the cells in each well per the following protocol: the medium/composition of the present invention was aspirated off; the cells were gently washed once with 300 μl of 1× Dulbecco's phosphate-buffered saline (DPBS); 300 μl of freshly prepared 4% parafonnaldehyde (PFA) in 1×DPBS was added; and the cells were fixed in 4% PFA for 15 minutes. The cells were then gently washed twice with 300 μl of 1×PBS; and then again twice with 300 μl of ddH2O. The cells were then stained with Alizarin red staining solution in the dark for 45 minutes; the stain then aspirated off; and the cells were gently washed again 2-3 times with 300 μl of ddH2O, which was subsequently aspirated off. Then 300 μl of 1×PBS was added, and the cells were imaged using a Nikon™ Eclipse TS100 inverted microscope with an AMscope camera at 4× (see FIGS. 2 and 3 images).

FIG. 2 are images of the results Alizarin red staining of four different compositions (replicated) after 14 days of culturing with 6,200 MSCs in each well. The four compositions are: box 1-Lonza™ Non-Differentiating Culture Medium (control); box 2-100 mg/ml of Cyplexinol® with Lonza™ Non-Differentiating Culture Medium; box 3-Lonza™ Osteogenesis Culture Medium (control); and box 4-Cyplexinol® with Lonza™ Osteogenesis Culture Medium.

At 14 days, the greatest amount of differentiation of MSC's to osteoblasts is shown by the red dots in the box 2's staining the osteoblast's bone mineral, which comprise the 100 mg/ml of Cyplexinol® with Lonza™ Non-Differentiating Culture Medium. By way of comparison, negligible MSC differentiation is shown in boxes 3 and 4 comprising, respectively, the Lonza™ Osteogenesis Culture Medium (control), and 100 mg/ml of Cyplexinol® with Lonza™ Osteogenesis Culture Medium.

FIG. 3 are images of the results Alizarin red staining of four different compositions (replicated) of FIG. 2 after 21 days of culturing with 6,200 MSCs in each well. Again, the greatest amount of differentiation of MSC's to osteoblasts is shown by the red dots in the box 2's staining of the osteoblast's bone mineral, which comprise the 100 mg/ml of Cyplexinol® with Lonza™ Non-Differentiating Culture Medium. Yet, at 21 days, the Lonza™ Osteogenesis Culture Medium (control), and 100 mg/ml of Cyplexinol® with Lonza™ Osteogenesis Culture Medium also display significant red staining.

### Exemplification 2—Osteogenic Gene Expression Analysis

Another set of experiments was performed to conduct gene expression testing of the bone formation for each of the four tested compositions: Lonza™ Non-Differentiating Culture Medium; 100 mg/ml of Cyplexinol® with Lonza™ Non-Differentiating Culture Medium; Lonza™ Osteogenesis Culture Medium; and 100 mg/ml of Cyplexinol® with Lonza™ Osteogenesis Culture Medium.

Gene expression protocol analysis comprised the following steps: plating 30,000 MSCs in each well of a 6-well plate; allowing 24 hours for cell attachment to the wells; adding one of the four tested compositions into each well; and changing the medium every 72 hours without damaging the cells. The RNA in the cells of each well are then collected using Qiagen™ RNeasy Plus Mini Kit, at 3, 7, and 10 days of cell growth/differentiation in the compositions.

The protocol was conducted in triplicate so that there were three identical 6 well plates tested (e.g. a total of 36 samples tested). For each sample, cDNA was synthesized using 100 ng of RNA and the Qiagen™ RT2 First Strand Kit. Quantitative (q-PCR) was employed using the Applied Biosystems™ 7900 HT Fast Real-time PCR System machine using five osteogenic primer pairs (e.g. for ALPL gene, SP7 gene, BGLAP gene, and GLI1 gene); and three housekeeping gene primer pairs (e.g. B2M housekeeping gene).

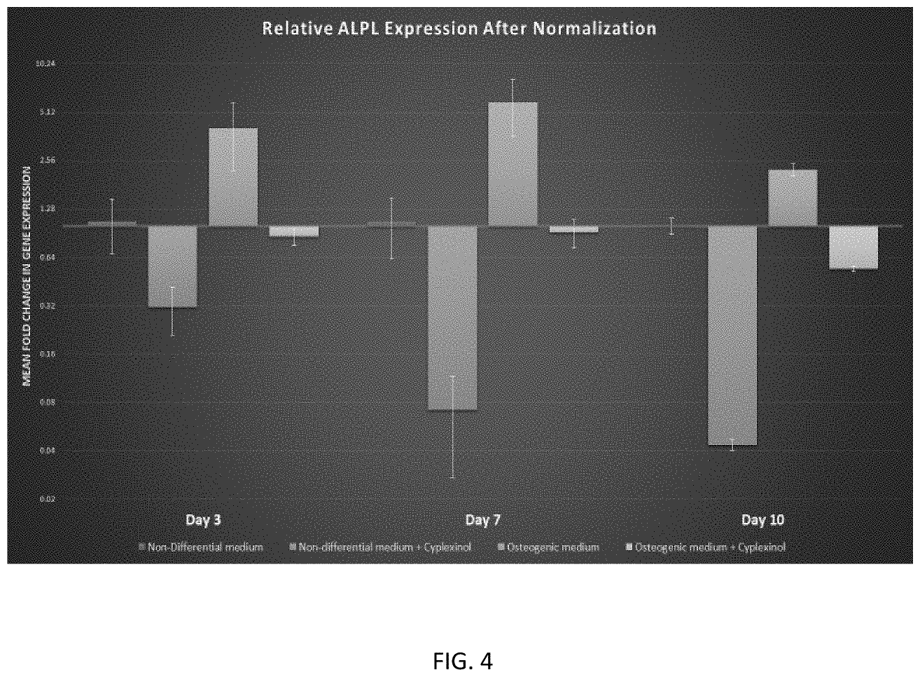

FIGS. 4-7 are bar charts illustrating the gene expression profiles tested in Experiment 3. Data Analysis was performed using the 2^{-AAct} method. The mean fold change for gene expression levels was graphed on a Log 2 scale; wherein a fold change greater than one represents upregulation of the gene with respect to the non-differentiating or osteogenic culture medium/composition; and a fold change of less than one represents downregulation of the gene with respect to the non-differentiating or osteogenic culture medium.

### ALPL Gene Expression

The ALPL protein is a tissue-nonspecific isozyme that is presumed to be involved in the calcification of bone matrix (5). It is encoded by the ALPL gene, thus ALPL gene expression is used herein as a marker for bone matrix/tissue formation.

FIG. 4 illustrate the change in ALPL expression levels at day 3, 7, 10. The greatest change was shown in the MSCs in the culture medium comprising the Lonza™ Non-differentiation Medium with Cyplexinol®. At day 3, the ALPL gene expression level change was reduced by 0.32; at day 7, it was reduced by 0.075, and at day 10 it was reduced by 0.045.

Results indicate that the culture medium comprising the Lonza™ Non-differentiation Medium and Cyplexinol® produced the greatest change in ALPL gene expression levels, and thus the largest amount of bone matrix production.

### BGLAP Gene Expression

As osteocalcin is produced by osteoblasts; and osteocalcin is encoded by the bone gamma-carboxyglutamic acid (BGLAP) gene. Herein, the change in BGLAP gene expression is used as a biomarker for bone formation.

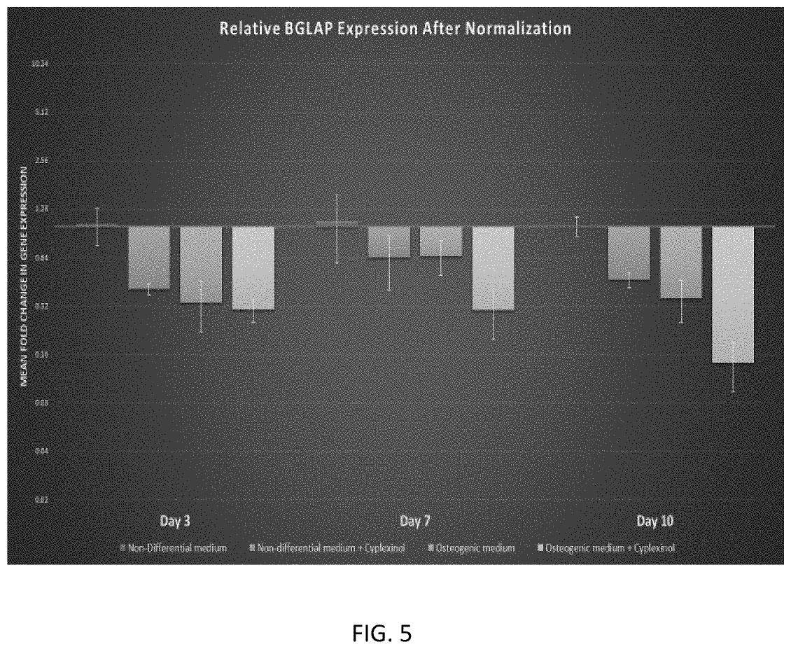

FIG. 5 illustrates the change in BGLAP gene expression levels at day 3, 7, 10. The greatest change in BGLAP gene expression levels was shown the Lonza™ Osteogenic Medium and Cyplexinol®.

### GLI1 Gene Expression

The Gli 1 marker is useful for the isolation, purification and identification of MSCs.

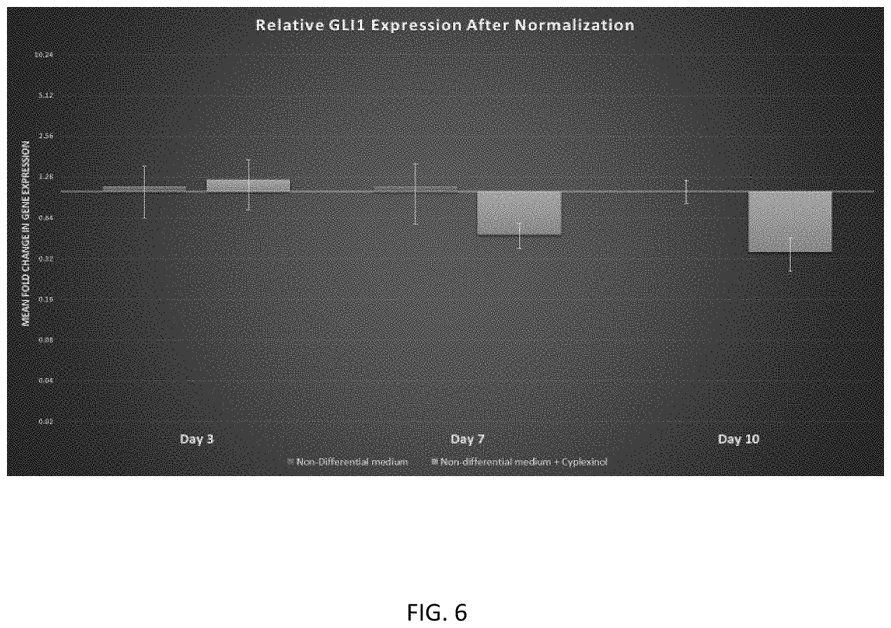

FIG. 6 illustrate the change in GLI1 gene expression levels at day 3, 7, and 10, with the Cyplexinol containing medium showing significant change, while the non-Cyplexinol containing medium showing little change in GLI1 gene expression levels, thus indicating that the culture medium comprising the Lonza™ Non-differentiation Medium and Cyplexinol® produced the largest reduction in MSCs (as they presumably differentiated to bone matrix/tissue).

### SP7 Gene Expression

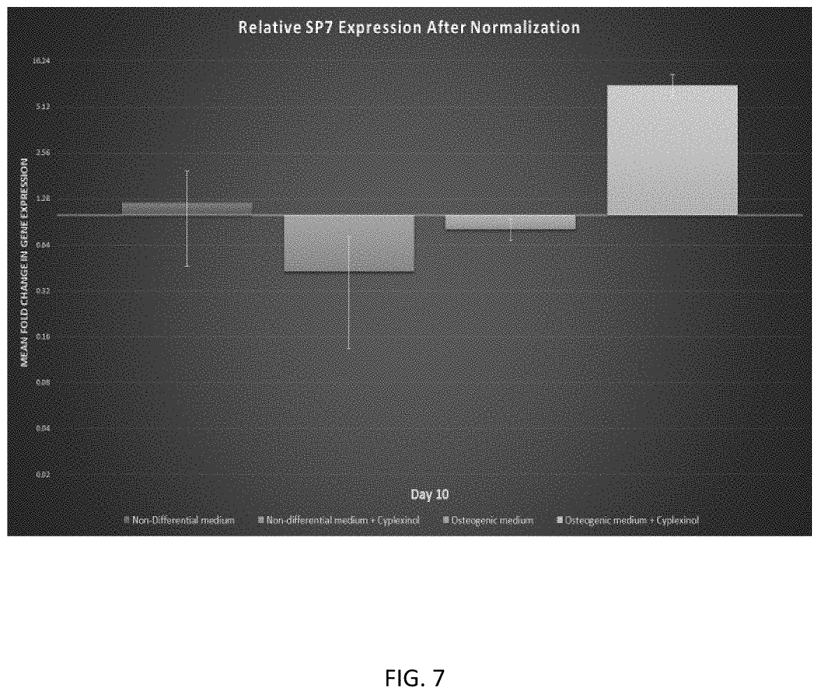

Sp7 protein (also known as osterix) induces MSCs to differentiate into osteoblasts, and subsequently osteocytes during bone formation. The Sp7 protein also plays a dual role to inhibit chondrocyte differentiation. FIG. 7 illustrates the change in Sp7 gene expression level at day 10.

Alternative Culture Conditions, Culture Mediums and/or Additional Culture Nutrients

Other culture media and conditions are envisioned within the scope of the present invention. In an embodiment, the MSCs may be cultured and expanded in suspension in a serum-free, essentially serum free, or serum reduced culture medium, the culture medium comprising: a media base; and a media supplement, such as by way of non-limiting examples: Cyplexinol® BMP complex; Lonza™ growth media (e.g. MSCGM-CD™ SingleQuots™ cryovials) dissolved in MSC basal medium. (e.g. see States Patent Application 20150329826 A1, that was filed on Dec. 12, 2013 by LONZA COLOGNE GMBH, entitled “MATERIALS AND METHODS FOR CELL CULTURE”.

In an embodiment, the MSCs are maintained in culture at optimal conditions for differentiation, such as 37° C., 5% CO2, 2-20% 02, and 95% humidity. The exact conditions can be adjusted by the skilled artisan as needed.

In addition to the composition comprising Cyplexinol® with Lonza™ osteogenic or non-differentiating growth media, the composition of the present invention may further comprise one or more cell growth and/or differentiation nutrients, such as by way of non-limiting examples: Dulbecco's Minimal Essential Medium (DMEM) supplemented with 10% fetal bovine serum (FBS), 1% penicillin-streptomycin, 100 nM dexamethasone, 10 mM beta-glycerophosphate, and 50 mM ascorbic acid-2-phosphate.

Basic fibroblast growth factor (bFGF, or FBG-2), transforming growth factors (TGF-(3), and bone morphogenetic proteins (e.g. BMP-2) are widely known in the art of tissue engineering as a supplement added to culture medium for MSCs. Alternatively, these proteins are endogenous to the Cyplexinol® demineralized bone and can signal MSCs to differentiate to osteoblasts, even in the absence of osteogenic culture medium (3). Additional supplements that may be added to the culture medium comprises, for example: human recombinant insulin, human recombinant PDGF-BB, and human recombinant EGF, in combination with human recombinant M-CSF and human recombinant FGF-2.

Alternative Culture Mediums suitable for use with Cyplexinol® in lieu of Lonza osteogenic or non-differentiating growth media, comprise, by way of non-limiting examples: Dulbecco's Modified Eagle Medium (DMEM), FBS 10%, 50 μg/ml ascorbic acid, 10 mM 0-glicerophosphate, 10 nM dexamethasone (optional); Fetal Bovine Serum, and Penicillin/Streptomycin/Amphotericin B solution, 100× or Penicillin/Streptomycin solution; or DMEM or the Eagle's Minimal Essential Medium in alpha-modification (alpha-MEM), supplemented with 10-20% fetal calf serum; or CellGro™ Hematopoietic Stem Cell medium with supplements comprising: human recombinant insulin, human recombinant PDGF-BB, and human recombinant EGF, in combination with human recombinant M-CSF and human recombinant FGF-2; or hBM-MSC Basal Medium with hBM-MSC Media Booster GTX (RoosterBio) with high glucose DMEM with L-Glutamine (Gibco, Carlsbad, Calif.), supplemented with 10% fetal bovine serum (PBS, Invitrogen™), 1% v/v antibiotic-antimycotic (Gibco), and 0.1 mM nonessential amino acids (Invitrogen™); or PromoCell™ MSC Differentiation Media.

The skilled artisan could readily derive the appropriate amount of Cyplexinol® power to add to the growth media, or culture medium, based on the experiments disclosed in FIGS. 1-7.

### Bioreactors

In addition to the MSCs in a laminar flow hood, the MSCs may be maintained in apparatus (e.g. a bioreactor) that provides optimal stirring, rocking or shaking conditions of the MSCs in culture medium for the reduction of shear stress levels and turbulences; and optimal nutrient delivery, temperature, changing of culture medium, gas levels, etc. Most bioreactors comprise a growth chamber having an inlet and an outlet and defining a cavity, a media reservoir, and a pump.

In an embodiment the bioreactor is used to produce a bone graft ex vivo for implantation (or to differentiate the MSCs sufficiently in the composition of the present invention) until the cells are of sufficient confluence, thickness, shape, stage of differentiation into bone, etc. to survive in vivo within a bone defect.

In another embodiment, MSCs are grown and differentiated into bone matrix within a bioreactor as a laboratory tool to study cell signaling, cell morphology, etc., and/or to study the effect of altering culture conditions on the cellular differentiation process and the ability to produce bone of sufficient mechanical strength comparable to endogenous bone.

Within the bioreactor, the MSCs may be grown and differentiated within and/or on a scaffold, such as a biocompatible, biodegradable scaffold, or while affixed to standard laboratory equipment (e.g. culture dishes, flasks, etc.) that are suitable to cellular experiments.

Furthermore, a bioreactor may be used to enclose the cells in the scaffold and/or cells in flasks or the like, in a sterile environment, while strictly controlling the MSC growth and differentiation to osteoblasts/bone (e.g. the perfusion of growth nutrients, temperature, pH, CO2 and 02 levels, etc.). Dense uniform cellular growth can be attained throughout the entire scaffold as a result of the composition/medium/growth nutrient perfusion. For example, the bioreactor can be used to maintain the MSCs at an: oxygen tension (or percentage of oxygen), between 0.33 mg/L and 7.1 mg/L of dissolved oxygen, adjustable by injecting nitrogen; the pH of the culture medium between 7.2 and 7.4, adjustable by adding a solution of a base, such as NaOH; and the temperature between 36.5° C. and 37.5° C.

Bioreactors for use in facilitating the proliferation and differentiation of MSCs to osteoblasts is well known in the art, such as the bioreactor disclosed in: U.S. Pat. No. 9,127,242 B2 that issued Sep. 8, 2015, and entitled “Tissue and organ graft bioreactor and method of operation”; and U.S. Pat. No. 9,260,686 B2 that issued Feb. 16, 2016, entitled “Tubular Bioreactor System for Use in Bone and Cartilage Tissue Engineering.”

Non-limiting examples of biocompatible polymer scaffolds to grow the MSCs on comprise, for example, polymers formed from materials such as: alginate beads comprising alginate, poly(caprolactone) (PCL), or poly(1-lactic acid) (PLLA); cellulose ether, cellulose, cellulosic ester, fluorinated polyethylene, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, and copolymers thereof, or physical blends thereof.

The biocompatible, biodegradable scaffold may also be impregnated with MSC differentiation and/or growth factors, stem cell factor (SCF), vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), insulin-like growth factor (IGF), cytokine growth factor (CGF), stem cell factor (SCF), colony stimulating factor (CSF), growth differentiation factor (GDF), integrin modulating factor (IMF), calmodulin (CaM), thymidinc kinase (TK), tumor necrosis factor (TNF), growth hormone (GH), bone morphogenic proteins (BMP), interferon, interleukins, cytokines, integrin, collagen, elastin, fibrillins. fibronectin, laminin, glycosaminoglycans, heparan sulfate, chondrotin sulfate (CS), hyaluronic acid (HA), vitronectin, proteoglycans, transferrin, cytotactin, tenascin, and lymphokines.

### Ex Vivo Bone Grafts

The present invention further comprises a method of producing a bone graft ex vivo using the composition disclosed herein to differentiate MSCs into a bone matrix for implantation into a bone defect within a patient (e.g. long or short bone, jaw, joint, etc.). The MSCs are autologous (e.g. derived from a patient's own bone marrow) in most cases to prevent graft rejection; or they are derived from a patient who is a match for HLA typing.

In an embodiment, the bone matrix is not fully formed ex vivo. Instead, the MSCs in the composition of the present invention are implanted into a biodegradable scaffold, allowed to partially differentiate to bone matrix, and then implanted in vivo, wherein endogenous growth nutrients and mechanical forces within the surgical tissue enable the scaffold to dissolve while the bone tissue forms to fill the surgical site hole.

In an embodiment, the MSCs are grown and differentiated within one of the compositions of the present invention (e.g. FIGS. 1-7), and then implanted in a scaffold (or grown on the scaffold), placed in a bioreactor, and perfused with the same composition, or another perfusion medium well known in the art for differentiating MSCs to osteoblasts of bone ex vivo.

The scaffolds may further be in the shape of the defect; or be housed within a mold shaped to fit the surgical site. In an embodiment, the bioreactor has a mold into which the composition of the present invention and/or other perfusion medium (e.g. nutrients) is pumped under pressure and ports at multiple sites through which the medium can enter and/or exit the mold. For example, see U.S. Pat. No. 9,687,348 B2, filed Mar. 3, 2010 and entitled “Method of making a personalized bone graft.”

The MSCs may be autologously derived, such as extracting approximately bone marrow blood from posterior iliac crests of the patient who is receiving the bone graft and/or seeded scaffold implantation (e.g. see United States Patent Application 20090305406 A1 published Dec. 10, 2019, and entitled “Method of cultivation of human mesenchymal stem cells, particularly for the treatment of non-healing fractures, and bioreactor for carrying out this cultivation method”).

### Kits

The present invention further comprises kits for shipping and storing the culture medium of the present invention. In an embodiment, the kits comprise demineralized bone matrix (e.g. Cyplexinol® powder or liquid) stored in a separate vial from a culture medium, such as the Lonza Non-differentiation Culture Medium, or the Lonza Osteogenic Culture Medium, or other culture media well known in the art for differentiating MSCs to osteoblasts. In another embodiment, the Cyplexinol® powder is premixed with the culture medium. The mesenchymal stem cells may be further shipped in a separate vial in the same kit, such as the Lonza™ MSCGM SingleQuots™ cryovials, or the like, although the medium and cells upon arrival are separated and stored at different temperatures.

In another embodiment, the kits comprise demineralized bone matrix (e.g. Cyplexinol® powder or liquid) stored in a separate vial from a culture medium with MSCs (cells may be in separate), with instructions for mixing; and separate vials for at least one pharmaceutical composition well known in the art for treating a bone related disease or disorder (e.g. osteoporosis); and instructions for administering both to a human patient

### Conclusion

It is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting.

The use of “including,” “comprising,” or “having” and variations thereof herein is meant to encompass the items listed thereafter and equivalents thereof as well as additional items. As used here and in the claims, singular articles such as “a” and “an” and “the” and similar references in the context of describing the elements (especially in the following claims) are to be construed to cover both the singular and the plural, unless otherwise indicated herein or clearly contradicted by context. Similarly, when the plural form is used it is to be construed to cover the singular form as the context permits. The use of any and all examples, or exemplary language (e.g., “such as”) is intended to better illuminate the embodiments and does not pose a limitation on the scope of the claims unless otherwise stated.

As used herein, the term “about” or “approximately” means within 20%, preferably within 10%, and more preferably within 5% of a given value or range.

Substantially: for purposes of the present invention, unless otherwise defined with respect to a specific property, characteristic or variable, the term “substantially” as applied to any criteria, such as a property, characteristic or variable, means to meet the stated criteria in such measure that one skilled in the art would understand that the benefit to be achieved, or the condition or property value desired is met.

While specific embodiments have been described, various changes and substitutions may be made without departing from the scope of the invention. Therefore, the invention described here should not be limited except by the following claims and their equivalents. Those skilled in the art will recognize, or be able to ascertain using no more than routine experimentation, many equivalents to the specific embodiments of the invention described herein. Such equivalents are intended to be encompassed by the following claims.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

### REFERENCES

(1) da Silva Meirelles L, Caplan A I, Nardi N B., Stem Cells 2008; 26(9):2287-99.
(2) Caplan A I., Cell Stem Cell 2008; 3(3):229-30.
(3) J. J. Scaffidi, K. F. Vieira, “Cyplexinol: A Natural BMP Complex with Osteoinductive and Anti-Inflammatory Activity Promotes De Novo Bone and Joint Tissue Growth”, Journal of Stem Cell Research & Therapy, (2017) 7(5): pages 1-5.
(4) H. Wang, B. Pang, Y. Li, D. Zhu, T. Pang, Y. Liu, “Dexamethasone has variable effects on mesenchymal stromal cells” Cytotherapy, (2012) (14 (4): 423-30.
(5) J. Liu, H. K. Nam, C. Campbell, K. C. D. Gasque, J. L. Milian, N. E. Hatch, “Tissue nonspecific alkaline phosphatase deficiency causes abnormal craniofacial bone development in the Alp1(−/−) mouse model of infantile hypophosphatasia”, Bone, (2014) 67: 81e94.
(6) E. Birmingham, G. L. Niebur, P. E. McHugh, G. Shaw, F. P. Barry, L. M. McNamara, “Osteogenic Differentiation of Mesenchymal Stem Cells is Regulated by Osteocyte and Osteoblast cells in a Simplified Bone Niche”, European Cells and Materials, (2012), 23.

1. A mesenchymal stem cell (MSC) culture medium composition comprising:
a) An isolated endogenous Bone Morphogenic Protein (BMP) complex comprising endogenous growth factors; and
b) wherein said culture medium is suitable for use in proliferating and differentiating mesenchymal stem cells cultured therein into osteoblasts.

2. The composition of claim 1, wherein the BMP complex comprises Cyplexinol® BMP complex.

3. The composition of claim 2, wherein the composition comprises 1 gram of Cyplexinol® to 9.25 ml of culture medium.

4. The composition of claim 1, wherein the BMP complex further comprises collagen type I and at least one member selected from the group consisting of TGF-02, TGF-01, BMP-2, BMP-3, BMP-4, BMP-5, BMP-6, BMP-7, BMP-8, BMP-9, BMP-10, IGF-I, IGF-11, bFGF and VEGF.

5. The composition of claim 2, wherein the culture medium comprises MSC Lonza non-differentiation culture medium, or a MSC Lonza osteogenic differentiation culture medium.

6. A method of making a mesenchymal stem cell culture medium composition, comprising:
a) adding an isolated endogenous bone morphogenic protein (BMP) complex into a culture medium comprising mesenchymal stem cells, at 1 gram powder per 9.25 millileters of medium in the composition;
b) rocking or shaking the composition for about two hours at about 4 degrees Celsius;
c) centrifuging the composition at 5,000×g for 5 minutes at 15 degrees Celsius to pellet an undissolved compound;
d) collecting a supernatant in a sterile syringe;
e) repeating steps (c-d) two or three times to collect the supernatant; and
f) pass the supernatant through a PES syringe filter.

7. The method of making of claim 6, wherein BMP complex comprises endogenous growth factors.

8. The method of making of claim 7, wherein the BMP complex further comprises collagen type I and collagen type II and at least one member selected from the group consisting of TGF-02, TGF-01, BMP-2, BMP-3, BMP-4, BMP-5, BMP-6, BMP-7, BMP-8, BMP-9, BMP-10, IGF-I, IGF-II, PDGF-AA, PDGF-BB, PDGF-AB, f3-FGF and VEGF.

9. The method of making of claim 6, wherein about 6,000 mesenchymal stem cells are added to each well of a plate before adding the composition.

10. A method of proliferating and differentiating a mesenchymal stem cell (MSC) culture medium into osteoblasts, comprising:
a) culturing mammalian mesenchymal stem cells in a cell culture medium capable of inducing differentiation of mammalian mesenchymal stem cells into osteoblasts;
b) wherein the culture medium comprises an isolated endogenous bone morphogenic (BMP) complex.

11. The method of claim 10, wherein the BMP complex is Cyplexinol® in powder form dissolved into the culture medium.

12. The method of claim 11, wherein the Cyplexinol powder is dissolved into a MSC Lonza Non-differentiating, or a MSC Lonza Osteogenic Culture Medium at 1 gram of Cyplexinol powder per 9.25 milliliters of medium.

13. The method of claim 11, wherein the MSCs are cultured within the medium for up to 28 days, and the culture medium is replaced with fresh culture medium comprising Cyplexinol powder every 72 (Original) hours.

14. The method of claim 10, wherein the MSCs are seeded on a scaffold or on a plurality of alginate beads, the scaffold or the beads are housed within a bioreactor, and the culture medium comprising the Cyplexinol powder dissolved therein is perfused through the bioreactor.

15. The method of claim 10, wherein BMP complex further comprises endogenous growth factors.

16. The method of claim 15, wherein the BMP complex further comprises collagen type I and at least one member selected from the group consisting of TGF-02, TGF-01, BMP-2, BMP-3, BMP-4, BMP-5, BMP-6, BMP-7, BMP-8, BMP-9, BMP-10, IGF-I, IGF-II, f3-FGF and VEGF.

17. A method of treating a bone defect in a mammal, comprising:
a) extracting and isolating autologous mesenchymal stem cells from a patient bone marrow or iliac crest, or providing immunologically matched heterologous stem cells;
b) culturing in vitro the mesenchymal stem cells in a cell culture medium capable of inducing differentiation of mammalian mesenchymal stem cells into bone matrix and/or osteoblasts, wherein the culture medium comprises bone morphogenic protein (BMP) complex dissolved into the culture medium; and
c) implanting the bone matrix, and/or osteoblasts, and/or culture medium comprising the mesenchymal stem cells, into a bone defect of the patient.

18. The method of claim 17, wherein the bone matrix is fully formed before implanting in vivo.

19. The method of claim 18, wherein the mesenchymal stem cells are seeded on a biocompatible, biodegradable scaffold, perfused with the culture medium comprising the BMP complex, and implanted into the bone defect, wherein the stem cells differentiate in vivo into a bone matrix.

20. The method of claim 19, wherein the scaffold is perfused with the culture medium within a laminar flow hood or a bioreactor in vitro, and/or within the bone defect in vivo.

21. The method of any claim 17, wherein the bone matrix formed is able to withstand in vivo normal mechanical forces applied to a human skeleton.

22. A method of treating a bone disorder, disease, or defect in a human patient, comprising:
a. providing a composition comprising an isolated endogenous bone morphogenic protein complex;
b. performing in vivo lavage and bone assay; and
c. administering the composition to the patient in need thereof.

23. The method of claim 22, wherein the BMP complex further comprises collagen type I and at least one member selected from the group consisting of TGF-02, TGF-01, BMP-2, BMP-3, BMP-4, BMP-5, BMP-6, BMP-7, BMP-8, BMP-9, BMP-10, IGF-I, IGF-II, f3-FGF and VEGF.24. The method of claim 22, wherein the composition further comprises mesenchymal stem cells (MSCs), and culture medium able to differentiate the MSCs to bone in vivo.

25. A method of treating a bone disorder, disease, or defect in a human patient, comprising:
a. performing a diagnostic assay detecting a level of bone morphogenetic proteins in a patient serum level;
b. determining if the BMP level is below a threshold level; and
c. when the BMP level is below the threshold level, administering a composition comprising isolated endogenous bone morphogenic protein complex with growth factors, able to differentiate in vivo into bone and/or osteoblasts.

26. The method of claim 25, wherein the BMP complex further comprises collagen type I and at least one member selected from the group consisting of TGF-02, TGF-01, BMP-2, BMP-3, BMP-4, BMP-5, BMP-6, BMP-7, BMP-8, BMP-9, BMP-10, IGF-I, IGF-II, f3-FGF and VEGF.

27. A method of treating a bone disorder, disease, or defect in a human patient, comprising administering a composition comprising isolated endogenous bone morphogenic protein complex with endogenous growth factors.

28. The method of claim 27, wherein the BMP complex further comprises collagen type I and at least one member selected from the group consisting of TGF-02, TGF-01, BMP-2, BMP-3, BMP-4, BMP-5, BMP-6, BMP-7, BMP-8, BMP-9, BMP-10, IGF-I, IGF-II, f3-FGF and VEGF.

29. The method of claim 27 wherein the composition is administered in conjunction with transplantation of heterologous or homologous stem cells.

30. The method of claim 27 wherein the composition is able to augment in vivo a naturally occurring differentiation of a plurality of endogenous stem cells to bone or osteoblasts.

31. The method of claim 27 wherein the composition is administered as an adjunct to a pharmaceutical treatment able to promote stem cell function.

32. The method of claim 31, wherein the pharmaceutical comprises an oral composition for the treatment of osteoporosis.
